SEQUENCE LISTING (15 sequences)

SEQ ID NO: 1 - protein, 9 aa, Artificial Sequence
  peptide
  MISC_FEATURE (1)..(1): Acp
XCGGSGGSC

SEQ ID NO: 2 - protein, 17 aa, Artificial Sequence
  peptide
  MISC_FEATURE (1)..(1): Acp
XCGGSGGSCRRRRRRRR

SEQ ID NO: 3 - protein, 30 aa, Artificial Sequence
  peptide
  MISC_FEATURE (1)..(1): Acp
  MISC_FEATURE (12)..(12): Aib
  MISC_FEATURE (15)..(15): Aib
  MISC_FEATURE (18)..(18): Aib
XCGGSGGSCLLXLLXLLXGGGRRRRRRRRR

SEQ ID NO: 4 - protein, 27 aa, Artificial Sequence
  peptide
  MISC_FEATURE (1)..(1): Acp
  MISC_FEATURE (12)..(12): Aib
  MISC_FEATURE (15)..(15): Aib
  MISC_FEATURE (18)..(18): Aib
XCGGSGGSCLLXLLXLLXGGGRRRRRR

SEQ ID NO: 5 - protein, 24 aa, Artificial Sequence
  peptide
  MISC_FEATURE (1)..(1): Acp
  MISC_FEATURE (12)..(12): Aib
XCGGSGGSCLLXGGGRRRRRRRRR

SEQ ID NO: 6 - protein, 21 aa, Artificial Sequence
  peptide
  MISC_FEATURE (1)..(1): Acp
  MISC_FEATURE (12)..(12): Aib
XCGGSGGSCLLXGGGRRRRRR

SEQ ID NO: 7 - protein, 18 aa, Artificial Sequence
  peptide
  MISC_FEATURE (1)..(1): Acp
  MISC_FEATURE (12)..(12): Aib
XCGGSGGSCLLXGGGRRR

SEQ ID NO: 8 - protein, 33 aa, Artificial Sequence
  peptide
  MISC_FEATURE (1)..(1): Acp
  MISC_FEATURE (12)..(12): Aib
  MISC_FEATURE (15)..(15): Aib
  MISC_FEATURE (18)..(18): Aib
  MISC_FEATURE (21)..(21): Aib
XCGGSGGSCLLXLLXLLXLLXGGGRRRRRRRRR

SEQ ID NO: 9 - protein, 19 aa, Artificial Sequence
  peptide
  MISC_FEATURE (1)..(1): Acp
  MISC_FEATURE (12)..(12): Aib
XCGGSGGSCLLXGRRRRRR

SEQ ID NO: 10 - protein, 22 aa, Artificial Sequence
  peptide
  MISC_FEATURE (1)..(1): Acp
  MISC_FEATURE (12)..(12): Aib
XCGGSGGSCLLXGGGRRRRRRR

SEQ ID NO: 11 - protein, 23 aa, Artificial Sequence
  peptide
  MISC_FEATURE (1)..(1): Acp
  MISC_FEATURE (12)..(12): Aib
  MISC_FEATURE (15)..(15): Aib
XCGGSGGSCLLXLLXGGGRRRRR

SEQ ID NO: 12 - protein, 24 aa, Artificial Sequence
  peptide
  MISC_FEATURE (1)..(1): Acp
  MISC_FEATURE (12)..(12): Aib
  MISC_FEATURE (15)..(15): Aib
XCGGSGGSCLLXLLXGGGRRRRRR

SEQ ID NO: 13 - protein, 26 aa, Artificial Sequence
  peptide
  MISC_FEATURE (1)..(1): Acp
  MISC_FEATURE (12)..(12): Aib
  MISC_FEATURE (15)..(15): Aib
  MISC_FEATURE (18)..(18): Aib
XCGGSGGSCLLXLLXLLXGGGRRRRR

SEQ ID NO: 14 - protein, 24 aa, Artificial Sequence
  peptide
  MISC_FEATURE (1)..(1): Acp
  MISC_FEATURE (24)..(24): Aib
XCGGSGGSCRRRRRRRRRGGGLLX

SEQ ID NO: 15 - protein, 24 aa, Artificial Sequence
  peptide
  MISC_FEATURE (1)..(1): Acp
XCGGSGGSCLLAGGGRRRRRRRRR

(19) United States
(12) Patent Application Publication
(10) Pub. No.: US 2022/0048953 A1
(43) Pub. Date: Feb. 17, 2022

(54) CELL-PENETRATING PEPTIDE

(71) Applicant: KANEKA CORPORATION, Osaka-shi, Osaka (JP)

(72) Inventors: Yuka MATSUDA, Takasago-shi (JP); Hiroshi KITA, Takasago-shi (JP); Keishi TAKATSU, Takasago-shi (JP); Tatsuya MOUTAI, Takasago-shi (JP); Mitsuaki KITANO, Takasago-shi (JP)

(73) Assignee: KANEKA CORPORATION, Osaka-shi, Osaka (JP)

(21) Appl. No.: 17/276,041

(22) PCT Filed: Aug. 9, 2019
(86) PCT No.: PCT/JP2019/031670

§ 371 Date: Mar. 12, 2021

(30) Foreign Application Priority Data
Sep. 26, 2018 (JP) 2018-180130

(51) Int. Cl. C07K 7/64; A61K 47/64

(52) U.S. Cl. CPC C07K 7/64; A61K 38/00; A61K 47/64

(57) ABSTRACT

Novel cell-penetrating peptides are provided, which are excellent in cell membrane permeability. The cell-penetrating peptide or salt thereof according to the present invention is characterized in comprising the sequence represented by formula (I) or the formula (II) as follows: X-(A-B-C)ₗ-(D)ₘ-(Arg)ₙ (Formula I) or X-(Arg)ₙ-(D)ₘ-(A-B-C)ₗ (Formula II), wherein X is a physiologically active peptide, A, B and C are aliphatic amino acids, D is an arbitrary amino acid, l is an integer of 1 or more and 4 or less, m is an integer of 0 or more and 5 or less, when l is 1, n is an integer of 8 or more, when l is 2, n is an integer of 6 or more, when l is 3, n is an integer of 4 or more, when l is 4, n is an integer of 4 or more.

### TECHNICAL FIELD

The present invention relates to a cell-penetrating peptide excellent in a cell membrane permeability.

### BACKGROUND ART

A molecularly targeted drug, which attacks a specific protein or gene as a target, has been recently attracted attention, and protein-protein interactions (PPI) is one of attractive drug discovery targets. On the one hand, it is difficult to inhibit PPI by an existing low molecular compound, since an interaction face between proteins is broad and highly hydrophilic. Though a middle molecular compound such as a peptide can inhibit PPI, many of the compounds do not have a cell membrane permeability.

As a method for delivering a peptide into a cell, a method by which a cell membrane permeability is given by binding a cell-penetrating peptide (CPPs) to a cargo molecule is known. As existing CPPs, TAT peptide derived from HIV-1 (Patent document 1), altered Penetrating derived from a homeodomain of drosophila Antennapedia (Patent document 2), and oligo-arginine (Non-patent documents 1 to 3) are known. In addition, Non-patent document 4 discloses various CPPs.

### PRIOR ART DOCUMENT

### Patent Document

Patent document 1: JP H10-33186 A

Patent document 2: JP 2002-530059 T

### Non-Patent Document

Non-patent document 1: Futaki, S. et al., J. Biol. Chem., 2001, 276, pp. 5836-5840

Non-patent document 2: Dana Maria Copolovici et al., ACS Nano, 2014, 8, p. 1972

Non-patent document 3: James R. Maiolo et al., Biochimica et Biophysica Acta, 1712 (2005), pp. 161-172

Non-patent document 4: Paul A. Wender et al., Proc. Natl. Acad. Sci., 2000, 97(24)8, pp. 13003-13008

### DISCLOSURE OF THE INVENTION

### Problems to be Solved by the Invention

Various cell-penetrating peptides have been known as described above; however, a better novel cell-penetrating peptide is searched, since there has been a few cell-penetrating peptides which are actually in clinical use.

Accordingly, the objective of the present invention is to provide a novel cell-penetrating peptide excellent in a cell membrane permeability.

### Means for Solving the Problems

The inventors of the present invention repeated intensive studies in order to solve the above-described problems. As a result, the inventors found a peptide having an excellent cell membrane permeability, and completed the present invention by finding that a physiologically active peptide can be efficiently delivered into a cell by binding such a peptide to the physiologically active peptide to be delivered into a cell.

The present invention is hereinafter described.

A cell-penetrating peptide or a salt thereof, comprising the sequence represented by the following formula (I) or the formula (II):

X−(A−B−C)ₗ−(D)ₘ−(Arg)ₙ (I)

X−(Arg)ₙ−(D)ₘ−(A−B−C)ₗ (II)

wherein

X is a physiologically active peptide,

A, B and C are independently aliphatic amino acids selected from alanine, 2-methylalanine, valine, leucine and isoleucine,

D is an arbitrary amino acid,

l is an integer of 1 or more and 4 or less,

m is an integer of 0 or more and 5 or less,

when l is 1, n is an integer of 8 or more,

when l is 2, n is an integer of 6 or more,

when l is 3, n is an integer of 4 or more,

when l is 4, n is an integer of 4 or more.

The cell-penetrating peptide or salt thereof according to the above [1], wherein the A and the B are leucines.

The cell-penetrating peptide or salt thereof according to the above [1] or [2], wherein the D is glycine.

The cell-penetrating peptide or salt thereof according to any one of the above [1] to [3], wherein the C is 2-methylalanine.

The cell-penetrating peptide or salt thereof according to any one of the above [1] to [4], wherein the physiologically active peptide is cyclized.

The cell-penetrating peptide or salt thereof according to any one of the above [1] to [5], wherein a C-terminus is amidated.

### Effect of the Invention

A physiologically active substance can be efficiently delivered into a cell by the cell-penetrating peptide of the present invention, since the present invention peptide has an excellent cell membrane permeability. The cell-penetrating peptide of the present invention is therefore very industrially superior, since the present invention peptide may become an excellent molecular targeted drug.

### BRIEF DESCRIPTION OF THE DRAWINGS

FIG. 1 is a graph to show logarithmic values of fluorescence intensity ratios obtained by a cell membrane permeability test of various peptide conjugates.

### MODE FOR CARRYING OUT THE INVENTION

The physiologically active peptide contained in the cell-penetrating peptide of the present invention is not particularly restricted as long as the physiologically active peptide is delivered into a cell to exhibit some sort of a physiological action in the cell. The number of an amino acid residue which constitutes the physiologically active peptide is preferably 4 or more and 20 or less, since the physiologically active peptide is delivered into a cell. Even if the number of the amino acid residues is 4, some physiologically active peptides show a physiological activity. When the number of the amino acid residue is 20 or less, the peptide may be delivered into a cell more surely. The number of the amino acid residue is more preferably 5 or more and 15 or less.

The physiologically active peptide may have a linker to bind to the cell membrane permeability-promoting peptide, i.e. (A-B-C)ₗ-(D)ₘ-(Arg)ₙ or (Arg)ₙ-(D)ₘ-(A-B-C)ₗ. The linker may be a general linker group in addition to an amino acid residue and a peptide. The linker group is not particularly restricted and is exemplified by a C₁₋₆ alkylene group, an amino group (—NH—), an imino group (>C═N— or —N═C<), an ether group (—O—), a thioether group (—S—), a carbonyl group (—C(═O)—), a thionyl group (—C(═S)—), an ester group (—C(═O)—O— or —O—C(═O)—), an amide group (—C(═O)—NH— or —NH—C(═O)—), a sulfoxide group (—S(═O)—), a sulfonyl group (—S(═O)₂—), a sulfonylamide group (—NH—S(═O)₂— and —S(═O)₂—NH—), and a group formed by binding 2 or more of the above groups. The bonding number of the linker group formed by binding 2 or more of the above groups is preferably 10 or less or 5 or less, and more preferably 3 or less. An example of the linker group formed by binding 2 or more of the above groups includes a C₁₋₆ alkylene group having an amino group, an imino group, an ether group, a thioether group, a carbonyl group, a thionyl group, an ester group, an amide group, a sulfoxide group, a sulfonyl group and/or a sulfonylamide group at the one end or the both ends. When the linker is a peptide, the number of the amino acid residue which constitutes the linker is preferably 1 or more and 20 or less. It is preferred that the linker peptide does not have an influence on the activity of the physiologically active peptide. An example of the linker peptide includes GS linker and GGS linker. The GGS linker has a sequence in which GGS sequence is repeated 1 time or more and about 6 times or less. The GS linker has a sequence in which GGGGS sequence is repeated 1 time or more and about 6 times or less, particularly 3 times.

The physiologically active peptide may be cyclized, if possible. When the physiologically active peptide is cyclized, the peptide is hardly attacked by a protease or the like in a living body and can be stabilized. In addition, a cell membrane permeability of such a cyclized peptide may be further improved. A side chain reactive group in the amino acid residue contained in the physiologically active peptide can be utilized for the cyclization. An example of the side chain reactive group includes a hydroxy group of Ser and Thr; a thiol group of Cys; a carboxy group of Asp and Glu; and an amino group of Lys.

A compound having a plurality of a reactive group which can be reacted with the side chain reactive group can be used as a crosslinking compound for the cyclization of the physiologically active peptide. The number of the reactive groups is preferably 2. An example of the reactive group includes a carboxy group, an active ester group, an acid chloride group, an acid bromide group, a halogeno group, an epoxy group, a hydroxy group and an amino group. A base, a condensation agent or the like may be added to accelerate a reaction for the cyclization.

An example of a linker group to bind a plurality of the reactive group in the crosslinking compound includes the above-described linker group to bind the physiologically active peptide to the N-terminal side part. The length of the linker group may be appropriately adjusted depending on the residue number between the amino acid residues to be utilized for the cyclization, a size of a desired ring or the like.

An example of the crosslinking compound to crosslink the physiologically active peptide includes the following compounds.

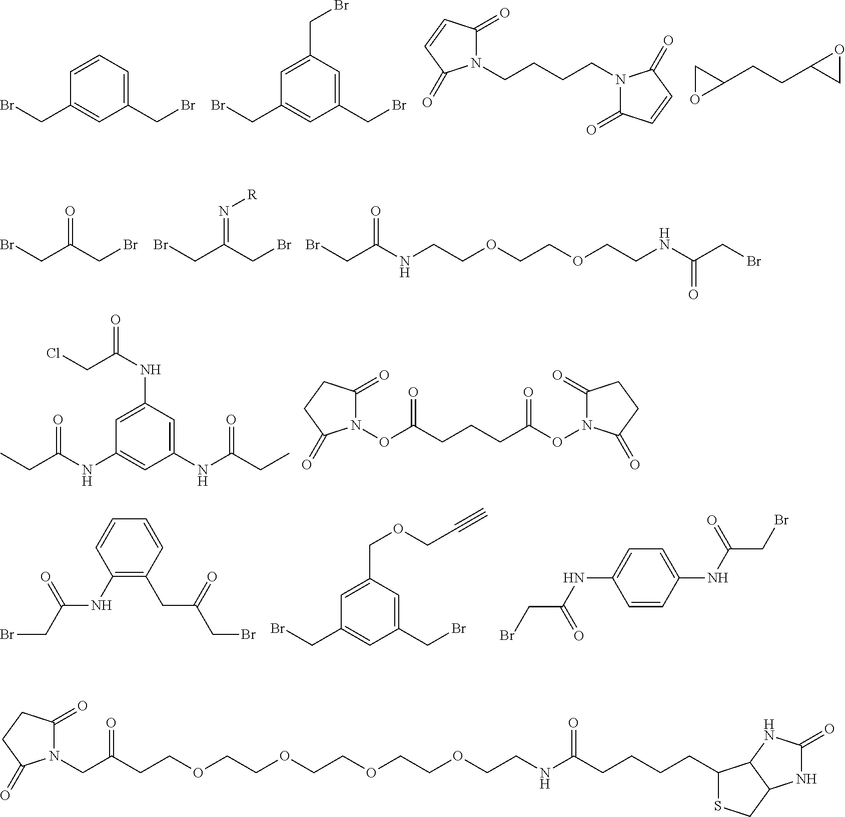

The N-terminal side of the cell-penetrating peptide according to the present invention is -(A-B-C)ₗ-(D)ₘ-(Arg)ₙ or -(Arg)ₙ-(D)ₘ-(A-B-C)ₗ. The peptides have a function to accelerate a cell membrane permeation of the physiologically active peptide. The peptides are conveniently referred to as a “cell membrane permeability-promoting peptide” in some cases.

The A to C in the cell membrane permeability-promoting peptide are independently aliphatic amino acids selected from alanine, 2-methylalanine, valine, leucine and isoleucine. The A and the B are preferably leucines, and the C is preferably alanine or 2-methylalanine, i.e. 2-aminoisobutyric acid, and more preferably 2-methylalanine.

It has been known that an [Arg] unit has a cell membrane permeability. A cell membrane permeability is remarkably improved in the present invention in comparison with an [Arg] unit by itself by using at least an [A-B-C] unit in addition to an [Arg] unit. The number of the [Arg] unit, i.e. “n”, is 4 or more, though the number is also dependent on the number of the [A-B-C] unit. With respect to the relation with the [A-B-C] unit, when the [A-B-C] unit number is smaller, the [Arg] unit number is preferably larger. Specifically, when the l as the number of the [A-B-C] unit is 1, the n is preferably an integer of 8 or more; when the l is 2, the n is preferably an integer of 6 or more; when the l is 3 or 4, the n is preferably an integer of 4 or more. The upper limit of the number of the [Arg] unit is not particularly restricted, and may be, for example, 16 or less, is preferably 14 or less or 12 or less, and more preferably 10 or less.

The [A-B-C] unit is a very important unit for a cell membrane permeability in the cell membrane permeability-promoting peptide. The present inventors experimentally found that a cell membrane permeability is remarkably improved by adding even one of the [A-B-C] unit to an oligo-arginine. Though the reason therefor is not necessarily clear, the secondary structure of the [A-B-C] unit may contribute the improvement of a cell membrane permeability as it is known a repeating sequence of [Leu-Leu-Aib] forms a helix structure. The number of the [A-B-C] unit, i.e. “l”, is 1 or more and 4 or less. The present inventors experimentally found that when there is not the [A-B-C] unit, the cell membrane permeability of the peptide is not sufficient at all. On the one hand, there are excessive [A-B-C] units, the peptide may become difficult to be handled due to a lower water solubility. The “l” is therefore preferably 4 or less.

The [D] unit in the cell membrane permeability-promoting peptide plays a role of a linker to mainly bind the [Arg] unit and the [A-B-C] unit. The D is an arbitrary amino acid, and is exemplified by Gly; Ala; a branched amino acid such as Val, Leu and Ile; a hydroxy amino acid such as Ser and Thr; a sulfur-containing amino acid such as Cys and Met; an acid amide amino acid such as Asn and Gln; Pro; an aromatic amino acid such as Phe, Thr and Trp; an acidic amino acid such as Asp and Glu; and a basic amino acid such as Lys, Arg and His. The D is preferably a neutral amino acid selected from Gly, Ala, a branched amino acid, a hydroxy amino acid, a sulfur-containing amino acid and an acid amide amino acid, more preferably an amino acid selected from Gly, Ala, Val, Leu and Ile, and even more preferably Gly. The number of the [D] unit, i.e. “m”, is 0 or more and 5 or less. The m is preferably 1 or more, more preferably 2 or more, and preferably 4 or less, more preferably 3 or less.

The position of (A-B-C)ₗ and the position of (Arg)ₙ in the cell-penetrating peptide of the present invention may be interchanged with each other, and the sequence represented by the formula (I) is more preferred.

For example, other peptide may be bound to the N-terminus or the C-terminus of the cell-penetrating peptide of the present invention as long as the cell-penetrating peptide has the sequence represented by the formula (I) or the formula (II). The other peptide bound to the terminus is not particularly restricted as long as the other peptide does not inhibit the cell membrane permeability of the present invention peptide, and for example, the number of the amino acid residue of the other peptide is preferably 1 or more and 10 or less and more preferably 5 or less. The sequence of the cell-penetrating peptide of the present invention is preferably composed of the sequence represented by the formula (I) or the formula (II) only and more preferably the sequence represented by the formula (I) only.

The N-terminus or the C-terminus of the cell-penetrating peptide according to the present invention may be chemically modified. For example, the C-terminus may be —COOH or —COO⁻, amidated (—CONH₂), alkylamidated (—CONHR) or esterified (—COOR). In addition, the N-terminus may be —NH₂ or —NH₃⁺ and acylated (—NHCOR). The R is a C₁₋₆ alkyl group. In particular, the C-terminus is preferably amidated. When the C-terminus is amidated, a resistance to exoprotease can be improved, and an intramolecular condensation reaction and an intermolecularly condensation reaction can be inhibited during a peptide synthesis.

The C₁₋₆ alkyl group means a linear or branched monovalent saturated aliphatic hydrocarbon group having a carbon number of 1 or more and 6 or less, and is exemplified by methyl, ethyl, n-propyl, isopropyl, n-butyl, isobutyl, s-butyl, t-butyl, n-pentyl and n-hexyl. The C₁₋₆ alkyl group is preferably a C₁₋₄ alkyl group, more preferably a C₁₋₂ alkyl group, and the most preferably methyl.

The cell-penetrating peptide of the present invention may be a salt. Such a salt is preferably pharmaceutically acceptable. An example of a counter cation which constitutes the salt includes a metal ion, an ammonium ion (NH₄⁺), an organic base ion and a basic amino acid ion. An example of the counter anion includes an inorganic acid ion, an organic acid ion and an acidic amino acid ion.

An example of the metal ion which constitutes the metal salt includes an alkali metal ion such as lithium ion, sodium ion and potassium ion; an alkaline earth metal ion such as calcium ion and barium ion; and magnesium ion. An example of the organic base which constitutes the organic base salt includes trimethylamine, triethylamine, pyridine, picoline, 2,6-lutidine, ethanolamine, diethanolamine, triethanolamine, cyclohexylamine, dicyclohexylamine and N,N′-dibenzylethylenediamine. An example of the basic amino acid which constitutes the basic amino acid salt includes lysine, arginine and histidine.

An example of the inorganic acid which constitutes the inorganic acid salt includes hydrochloric acid, hydrobromic acid, nitric acid, sulfuric acid and phosphoric acid. An example of the organic acid which constitutes the organic acid salt includes formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid and p-toluenesulfonic acid. An example of the acidic amino acid which constitutes the acidic amino acid salt includes aspartic acid and glutamic acid.

The cell-penetrating peptide of the present invention can be produced by an ordinary method. For example, the cell-penetrating peptide can be produced by a solid-phase synthesis method, since a total amino acid residue number thereof is relatively small. Specifically, an amino acid sequence of the cell-penetrating peptide is designed, the C-terminal amino acid residue of which amino group and optionally a side chain reactive group are protected is bound to a solid resin, afterward a deprotection of the amino group and a binding of the next amino acid residue are repeated, and finally the peptide is cleaved from the solid resin and deprotected. Washing is conducted after each reaction.

When the physiologically active peptide is cyclized, the peptide bound to a solid resin may be cyclized or the peptide cleaved from a solid resin may be cyclized. It is preferred that the peptide cleaved from a solid resin is cyclized, since the number of production steps is smaller. Since the cell membrane permeability-promoting peptide is composed of amino acid residues which do not have a reactive group in a side chain, the physiologically active peptide may be basically cyclized by a crosslinking compound.

Since the physiologically active peptide gets through a cell membrane and is delivered into a cell by the cell-penetrating peptide of the present invention, the function and effect of the physiologically active peptide may be effectively exerted with relatively few side effects. The cell-penetrating peptide of the present invention is preferably injected to be administered, since the cell-penetrating peptide is a peptide.

A solvent of an injection product containing the cell-penetrating peptide of the present invention is preferably water. The injection product may further contain a water-miscible organic solvent such as ethanol, ethylene glycol, propylene glycol and polyethyleneglycol depending on a water solubility of the present invention peptide. In addition, the injection product may contain a salt such as sodium chloride, a buffer constituent and a preservative. Needless to say, the injection product is needed to be an isotonic fluid or a nearly isotonic fluid.

A dosage amount of the cell-penetrating peptide according to the present invention may be appropriately adjusted depending on a severity, age, sex, weight, symptom or the like of a patient to whom the cell-penetrating peptide is administered. For example, the dosage amount may be adjusted in the range of 0.001 mg/kg/day or more and 100 mg/kg/day or less, and preferably 0.005 mg/kg/day or more and 50 mg/kg/day or less.

The present application claims the benefit of the priority date of Japanese patent application No. 2018-180130 filed on Sep. 26, 2018. All of the contents of the Japanese patent application No. 2018-180130 filed on Sep. 26, 2018, are incorporated by reference herein.

### EXAMPLES

Hereinafter, the examples are described to demonstrate the present invention more specifically, but the present invention is in no way restricted by the examples, and the examples can be appropriately modified to be carried out within a range which adapts to the contents of this specification. Such a modified example is also included in the range of the present invention.

### Examples 1 to 9 and Comparative Examples 1 to 6: Synthesis of Peptide Conjugate

A peptide chain part of a peptide conjugate having the following sequence was synthesized on Rink Amide resin (0.2 mmol/g) by a solid-phase synthesis method using a microwave.

F-Ahx-(cargo peptide)-(Leu-Leu-Aib)ₗ-(Gly)ₘ-(Arg)ₙ-NH₂ wherein F is a fluorescein-containing group as a fluorescent group, Ahx is 6-aminohexanoic acid, Aib is 2-aminoisobutyric acid, i.e. 2-methylalanine, and cargo peptide has the following structure.

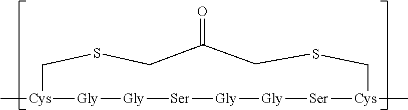

TABLE 1

| | Sequence | | | |
|---|---|---|---|---|
| | number | l | m | n |
| Comparative | 1 | 0 | 0 | 0 |
| example 1 | | | | |
| Comparative | 2 | 0 | 0 | 8 |
| example 2 | | | | |
| Example 1 | 3 | 3 | 3 | 9 |
| Example 2 | 4 | 3 | 3 | 6 |
| Example 3 | 5 | 1 | 3 | 9 |
| Comparative | 6 | 1 | 3 | 6 |
| example 3 | | | | |
| Comparative | 7 | 1 | 3 | 3 |
| example 4 | | | | |
| Example 4 | 8 | 4 | 3 | 9 |
| Example 5 | 9 | 1 | 0 | 9 |
| Comparative | 10 | 1 | 3 | 7 |
| example 5 | | | | |
| Comparative | 11 | 2 | 3 | 5 |
| example 6 | | | | |
| Example 6 | 12 | 2 | 3 | 6 |
| Example 7 | 13 | 3 | 3 | 5 |

### Example 8 (SEQ ID NO: 14): F-Ahx-(cargo peptide)-(Arg)₉-(Gly)₃-(Leu-Leu-Aib)-NH₂

### Example 9 (SEQ ID NO: 15): F-Ahx-(cargo peptide)-(Leu-Leu-Ala)-(Gly)₃-(Arg)₉-NH₂

A resin on which a peptide was synthesized was immersed in a mixed solution of trifluoroacetic acid (TFA)/water/triisopropylsilane (TIS)/3,6-dioxa-1,8-octanedithiol (DODT)=92.5/2.5/2.5/2.5 (by volume) for 3 hours, and the peptide was separated from the resin.

The obtained peptide was dissolved in a mixed solvent of N,N-dimethylformamide (DMF) and water, and the solution was treated with 1.5 equivalents of 1,3-dibromoacetone and 3.0 equivalents of N,N-diisopropylethylamine for 1 hour to cyclize the cargo peptide.

The peptide was purified from the reaction solution by reverse phase HPLC and freeze-dried. Then, the peptide was treated with 1.5 equivalents of fluoresceinisothiocyanate and 3.0 equivalents of N,N-diisopropylethylamine in DMF for 4 hours to fluorescently label the N-terminus. Next, the peptide was purified by reverse phase HPLC to synthesize the peptide conjugates of Examples 1 to 9 and Comparative examples 1 to 6.

Test example 1: Cell Membrane Penetrating Performance Evaluation

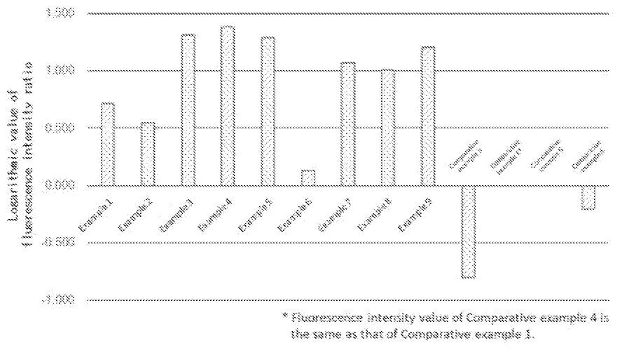

HeLa cell (Human cervix adenocarcinoma cell) was cultivated in a culture fluid containing 2 μM of the peptide conjugate of Examples 1 to 9 or Comparative examples 1 to 6 at 37° C. for 2 hours. Then, the cell was collected and stained with a propidium iodide solution to measure a fluorescence intensity using a flow cytometer, and a fluorescence intensity ratio to Comparative example 2 was calculated in accordance with the following formula. The result is shown in FIG. 1 and Table 2.

Fluorescence intensity ratio=(Fₙ−F₁)/(F₂−F₁)

Fₙ: Fluorescence intensity mode value of tested compound

F₁: Fluorescence intensity mode value of tested compound of Comparative example 1

F₂: Fluorescence intensity mode value of tested compound of Comparative example 2

TABLE 2

| | Fluorescence | | Fluorescence |
|---|---|---|---|
| | intensity ratio | | intensity ratio |
| Example 1 | 2.05 | Comparative | 1.00 |
| | | example 2 | |
| Example 2 | 1.72 | Comparative | 0.45 |
| | | example 3 | |
| Example 3 | 3.73 | Comparative | 0.00 |
| | | example 4 | |
| Example 4 | 4.00 | Comparative | 1.00 |
| | | example 5 | |
| Example 5 | 3.63 | Comparative | 0.81 |
| | | example 6 | |
| Example 6 | 1.14 | | |
| Example 7 | 2.92 | | |
| Example 8 | 2.74 | | |
| Example 9 | 3.34 | | |

As the result shown in FIG. 1 and Table 2, the fluorescence intensities 2 hours after the cultivation to Comparative example 2 were compared; as a result, it was experimentally demonstrated that the peptide conjugates of Examples 1 to 9 according to the present invention are excellent in cell membrane penetrating performance in comparison with that of Comparative example 2 which has a [Arg] unit chain but which does not have a [Leu-Leu-Aib] unit, though it has been taken for granted that the [Arg] unit has a cell membrane permeability.

1. A cell-penetrating peptide or a salt thereof, comprising the sequence represented by the following formula (I) or the formula (II):
X−(A−B−C)ₗ−(D)ₘ−(Arg)ₙ (I)
X−(Arg)ₙ−(D)ₘ−(A−B−C)ₗ (II)
wherein
X is a physiologically active peptide,
A, B and C are independently aliphatic amino acids selected from alanine, 2-methylalanine, valine, leucine and isoleucine,
D is an arbitrary amino acid,
l is an integer of 1 or more and 4 or less,
m is an integer of 0 or more and 5 or less,
when l is 1, n is an integer of 8 or more,
when l is 2, n is an integer of 6 or more,
when l is 3, n is an integer of 4 or more,
when l is 4, n is an integer of 4 or more.

2. The cell-penetrating peptide or salt thereof according to claim 1, wherein the A and the B are each leucine.

3. The cell-penetrating peptide or salt thereof according to claim 1, wherein the D is glycine.

4. The cell-penetrating peptide or salt thereof according to claim 1, wherein the C is 2-methylalanine.

5. The cell-penetrating peptide or salt thereof according to claim 1, wherein the physiologically active peptide is cyclized.

6. The cell-penetrating peptide or salt thereof according to claim 1, wherein a C-terminus is amidated.

7. The cell-penetrating peptide or salt thereof according to claim 1, wherein the number of amino acid residues in the physiologically active peptide is 4 or more, and 20 or less.

8. The cell-penetrating peptide or salt thereof according to claim 1, wherein the number of amino acid residues in the physiologically active peptide is 5 or more, and 20 or less.

9. The cell-penetrating peptide or salt thereof according to claim 1, wherein the number of amino acid residues in the physiologically active peptide is 5 or more, and 15 or less.

10. The cell-penetrating peptide or salt thereof according to claim 1, wherein the cell-penetrating peptide or a salt thereof comprises the following sequence:
X−(Leu−Leu−Aib)ₗ−(Gly)ₘ−(Arg)ₙ−NH₂
wherein
X is a physiologically active peptide,
l is an integer of 1 or more and 4 or less,
m is an integer of 0 or more and 5 or less,
when l is 1, n is an integer of 8 or more,
when l is 2, n is an integer of 6 or more,
when l is 3, n is an integer of 4 or more,
when l is 4, n is an integer of 4 or more.

11. The cell-penetrating peptide or salt thereof according to claim 10, wherein the cell-penetrating peptide or a salt thereof comprises the following sequence:
X−(Leu−Leu−Aib)₃−(Gly)₃−(Arg)₉−NH₂;
X−(Leu−Leu−Aib)₃−(Gly)₃−(Arg)₆−NH₂;
X−(Leu−Leu−Aib)−(Gly)₃−(Arg)₉−NH₂;
X−(Leu−Leu−Aib)₄−(Gly)₃−(Arg)₉−NH₂;
X−(Leu−Leu−Aib)−(Arg)₉−NH₂;
X−(Leu−Leu−Aib)₂−(Gly)₃−(Arg)₆−NH₂;
X−(Leu−Leu−Aib)₃−(Gly)₃−(Arg)₅−NH₂;
X−(Arg)₉−(Gly)₃−(Leu−Leu−Aib)−NH₂; and
X−(Leu−Leu−Ala)−(Gly)₃−(Arg)₉−NH₂.

12. A pharmaceutical product comprising an effective dosage amount of the cell-penetrating peptide or salt thereof according to claim 1.

13. A pharmaceutical product according to claim 12, wherein the product is formulated for injection.

14. A pharmaceutical product according to claim 14, wherein the product is formulated with a water-miscible organic solvent selected from the group consisting of ethanol, ethylene glycol, propylene glycol and polyethyleneglycol.

15. A pharmaceutical product according to claim 15, wherein the product further comprises a salt, a buffer constituent and a preservative.

16. A method of treatment, comprising administering an effective dosage amount of the cell-penetrating peptide or salt thereof according to claim 1 to a patient.

17. A method of treatment, comprising administering an effective dosage amount of the pharmaceutical product according to claim 12 to a patient.

18. A method of treatment, comprising administering an effective dosage amount of the pharmaceutical product according to claim 13 to a patient.
